(19) United States
(12) Patent Application Publication
(10) Pub. No.: US 2020/0040018 A1
(43) Pub. Date: Feb. 6, 2020

(54) AMINE SYNERGISTS

(71) Applicant: Hewlett-Packard Development Company, L.P., Spring, TX (US)

(72) Inventor: Zhang-Lin ZHOU, San Diego, CA (US)

(73) Assignee: Hewlett-Packard Development Company, L.P., Spring, TX (US)

(21) Appl. No.: 16/339,254

(22) PCT Filed: Jan. 31, 2017
(86) PCT No.: PCT/US2017/015776

§ 371 Date: Apr. 3, 2019

(51) Int. Cl. C07F 9/53; C07C 215/12; C07C 309/14; C09D 11/101; C09D 11/38

(52) U.S. Cl. CPC C07F 9/5337; C07C 215/12; C09D 11/38; C09D 11/101; C07C 309/14

(57) ABSTRACT

The present disclosure is drawn to an amine synergist including a tertiary benzylamine with a substituted benzene ring attached to a tertiary amine. The tertiary amine can be modified with multiple water soluble groups.

### BACKGROUND

Recently, curing of ink by radiation, and in particular ultraviolet (UV) curing, has become popular. UV curable ink can be cured after printing by application of UV light. Typically, UV curable inks include monomers that form polymers by free radical polymerization. The growing end of each polymer chain is a radical that reacts with additional monomers, transferring the radical to the end of the chain as each monomer is added. A photo initiator is used to form the first radicals to begin the polymerization process. The photo initiator is capable of absorbing UV light to generate radicals to react with the monomers.

Two types of photo initiators can be used in UV curable compositions. Type I photo initiators are unimolecular photo initiators that undergo a hemolytic bond cleavage upon absorption of UV light, forming radicals. Type II photo initiators are bimolecular photo initiators. These are used as a system of a photo initiator with a synergist, which can together form radicals upon exposure to UV light. Some Type II photo initiators react by hydrogen abstraction from the synergist to the photo initiator.

### BRIEF DESCRIPTION OF THE DRAWINGS

FIG. 1 is an example general scheme depicting synthesis of a benzyl tertiary amine of the present disclosure; and

FIG. 2 depicts an example method of preparing a photo curable ink in accordance with examples of the present disclosure.

### DETAILED DESCRIPTION

Most commercially available photo initiators that absorb LED light are not water soluble, thus they are typically not suitable for use in water based inks. One solution is to disperse these types of compounds, but there still remain issues with respect to crystallization at higher concentrations in the ink. With specific reference to Type II photo initiators, amine synergists can be used to achieve cross-linking polymerization of the reactive binders. In accordance with this, the present disclosure provides amine synergists that can be used for Type II photo-polymerization, and which are water soluble. These amine synergists are also chemical stable in aqueous solutions. In one example, these amine synergists can include a benzene ring with tertiary amines attached thereto modified with a water soluble group. Thus, the amine synergists can be water soluble and stable in aqueous inks, such as aqueous thermal inkjet ink.

The inkjet printing industry uses various types of inks, such as oil-based inks, solvent-based (non-aqueous) inks, water-based (aqueous) inks, and solid inks which are melted in preparation for dispensing. Solvent-based inks are fast drying, and as a result, are widely used for industrial printing. When solvent-based inks containing binders and other ingredients are jetted onto a substrate, the solvent(s) partially or fully evaporate from the ink, leaving the binder and other ingredients such as pigment particles on the printed substrate in the form of a dry film. During the drying process, the solvents, which are often volatile organic compounds (VOC), emit vapors, and therefore, can pollute the environment. The amount of pollution produced can increase greatly with higher printing speeds or for wide format images, where large amounts of ink are deposited onto a substrate. As a result of this and other concerns, efforts related to preparing inks that are environmentally friendly have moved some research in the direction of water-based inks. However, radiation-curable (or photo-curable) water-based ink compositions are noticeably limited among available options due to their specific formulation properties. Accordingly, the development of radiation-curable water-based inks, otherwise referred to as photo curable water-based inks, that exhibit specific desirable printing properties such as, for example, jetting properties as well as improved adhesion, would be desirable in the field of inkjet technology.

Accordingly, an amine synergist can include a tertiary benzylamine including a substituted benzene ring attached to a tertiary amine, wherein the tertiary amine is modified with multiple water soluble groups. In one example, the multiple water soluble groups can be the same group, or can be different groups. Suitable water soluble groups that can be used include C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups. In another example, the substituted benzene ring can be provided by a diphenyl(2,4,6-trimethylbenzoyl)phosphine oxide (e.g., with one or two water soluble tertiary amines). In another example, the substituted benzene ring can be provided by multi(halomethyl)benzene, such as a bis(chloromethyl)benzene (e.g., ortho, meta, or para), or a tris(chloromethyl)benzene.

In another example, a photo curable ink can include a photo reactive binder; an amine synergist including a tertiary benzylamine including a substituted benzene ring attached to a tertiary amine, wherein the tertiary amine is modified with multiple water soluble groups; and a Type II photo initiator. The photo curable ink can further include a colorant; and a liquid vehicle including co-solvent and water. The photo curable ink can have a pH from 7 to 12 and the amine synergist can be stable in the photo curable ink. In one example, the Type II photo initiator can be a polymeric photo initiator and the photo curable ink can further include a co-photo initiator. With respect to the amine synergist, in one example, the multiple water soluble groups can be the same group, or can be different groups. Suitable water soluble groups that can be used include C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups. In another example, the substituted benzene ring can be provided by a diphenyl(2,4,6-trimethylbenzoyl)phosphine oxide (e.g., with one or two water soluble tertiary amines). In another example, the substituted benzene ring can be provided by a multi(halomethyl)benzene, such as a bis(chloromethyl)benzene (e.g., ortho, meta, or para), or a tris(chloromethyl)benzene.

In another example, a method of making a photo curable ink can include mixing a photo reactive binder, a Type II photo initiator, a colorant, and a liquid vehicle including co-solvent and water with a tertiary benzylamine including a substituted benzene ring attached to a tertiary amine. The tertiary amine can also be modified with multiple water soluble groups. In one example, the one or more of the water soluble groups of the amine synergist is C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups. In another example, the substituted benzene ring can be provided by a diphenyl(2,4,6-trimethylbenzoyl)phosphine oxide (e.g., with one or two water soluble tertiary amines). In another example, the substituted benzene ring can be provided by multi(halomethyl)benzene, such as a bis(chloromethyl)benzene (e.g., ortho, meta, or para), or a tris(chloromethyl)benzene.

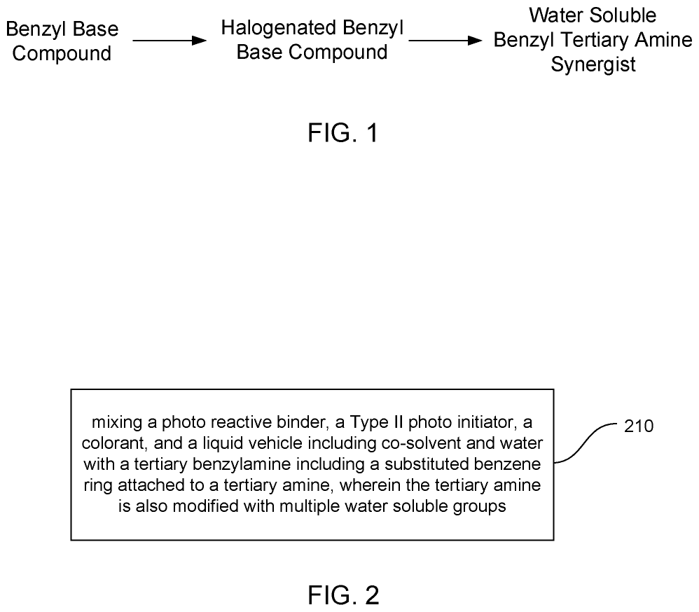

In further detail, a photo-initiator is one component found in UV-LED curable inks, which absorbs light and produces primary radical species and then initiate polymerization of curable binders in the ink. Based on their radical generation mechanisms, photo-initiators are divided into cleavage-type (Type I) and hydrogen abstraction type (Type II). Type I photo initiators include initiating species generated through highly efficient alpha cleavage. Type II photo initiators include initiating species generated through hydrogen abstraction from donor molecules (the amine synergist). Thus, for Type II photo initiators to work, donor molecules are present to achieve cross-linking polymerization of the reactive binders. These donor molecules of the present disclosure include tertiary amine based molecules that have been modified to include water soluble groups. The formation of the water soluble benzyl tertiary amines is shown generally in FIG. 1.

In accordance with this, the present disclosure is drawn to an amine synergist, including a tertiary benzylamine including a substituted benzene ring attached to a tertiary amine, wherein the tertiary amine is modified with multiple water soluble groups. The water soluble groups can include one or more of C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups. The substituted benzene ring can, in one example, be provided by a diphenyl(2,4,6-trimethylbenzoyl)phosphine oxide.

In another example, a photo curable ink can include a photo reactive binder; an amine synergist including a tertiary benzylamine including a substituted benzene ring attached to a tertiary amine, wherein the tertiary amine is modified with multiple water soluble groups; a Type II photo initiator; a colorant; and a liquid vehicle including co-solvent and water. In one example, the photo curable ink can have a pH from 7 to 12 and the amine synergist can be stable in the photo curable ink. In another example, the Type II photo initiator can be a polymeric photo initiator and the photo curable ink can further include a co-photo initiator. The water soluble group or groups of the amine synergist can be C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups. In one example, the substituted benzene ring can be provided by a diphenyl(2,4,6-trimethylbenzoyl)phosphine oxide.

In another example, a method of making a photo curable ink can include mixing a photo reactive binder, a Type II photo initiator, a colorant, and a liquid vehicle including co-solvent and water with a tertiary benzylamine including a substituted benzene ring attached to a tertiary amine. The tertiary amine can be also modified with multiple water soluble groups. The water soluble groups can be independently one or more of C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups. In one example, the substituted benzene ring can be part of or provided by a diphenyl(2,4,6-trimethylbenzoyl)phosphine oxide.

In accordance with the present disclosure, the amine synergist can have General Formula 1, as follows:

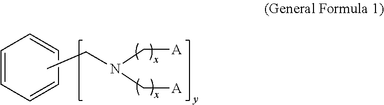

wherein A is independently a water solubilizing group, x is independently from 2 to 5, and y is from 1 to 4. Thus, each A can independently be hydroxyl, sulfonate, sulfonic acid, carboxylate, carboxylic acid, or polyethylene glycol with from 2 to 20 ether groups, for example.

In another example, the amine synergist can have General Formula 2 or General Formula 3, as follows:

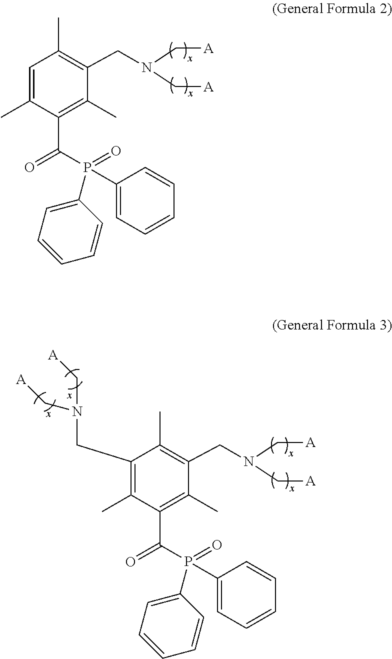

wherein A is independently a water solubilizing groups, and x is independently from 2 to 5. Thus, for example, A can be hydroxyl, sulfonate, sulfonic acid, carboxylate, carboxylic acid, or polyethylene glycol with from 2 to 20 ether groups.

Formulas I to XVIII below provide 18 examples of water soluble amine synergists that can be used in accordance with examples of the present disclosure. By way of specific example, these 18 example structures relate to General Formulas 1-3 where A is any of a number of water soluble groups, as shown below, and where x is generally 2 (ethyl) and y is 2. In the structures shown below, n can be from 1 to 100; and M can be hydrogen or a monovalent cation such as an alkali metal, e.g., sodium, potassium, etc., or ammonium).

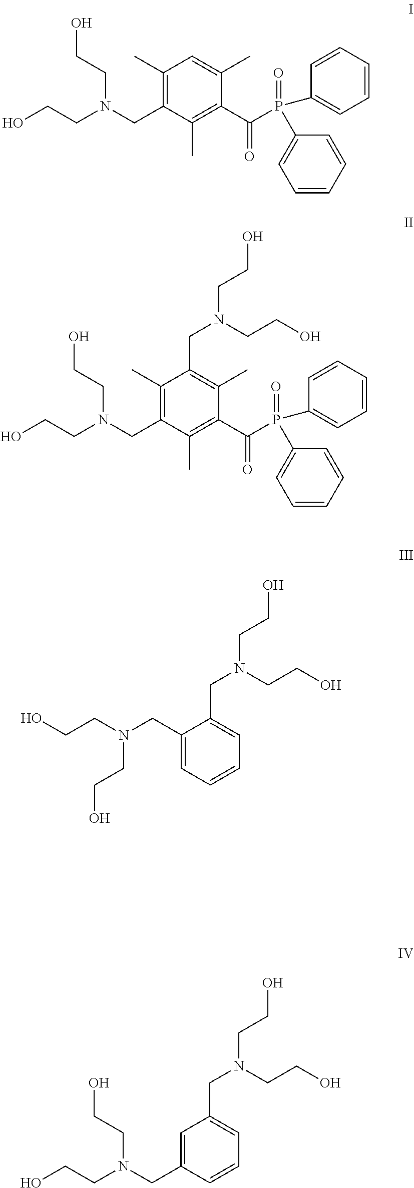 [SMILES: Cc1cc(C)c(C(=O)P(=O)(c2ccccc2)c2ccccc2)c(C)c1CN(CCO)CCO ; Cc1c(CN(CCO)CCO)c(C)c(C(=O)P(=O)(c2ccccc2)c2ccccc2)c(C)c1CN(CCO)CCO ; OCCN(CCO)Cc1ccccc1CN(CCO)CCO ; OCCN(CCO)Cc1cccc(CN(CCO)CCO)c1]

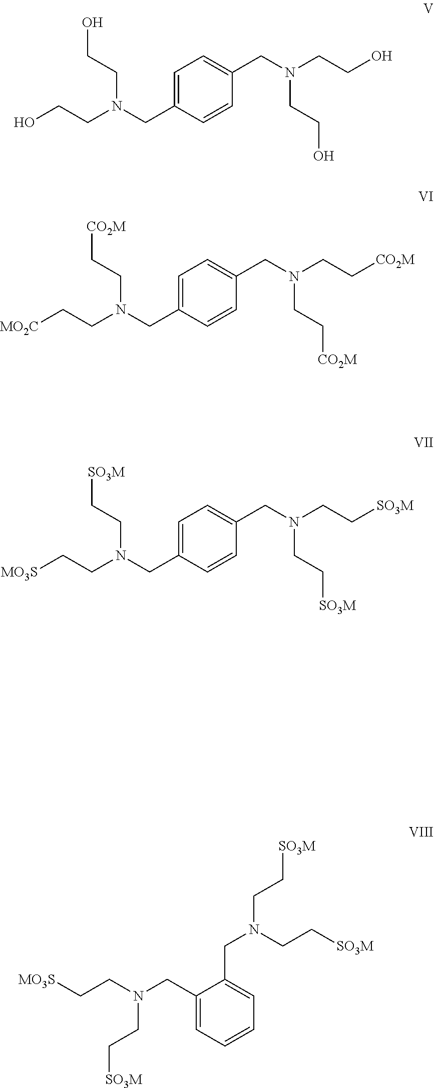

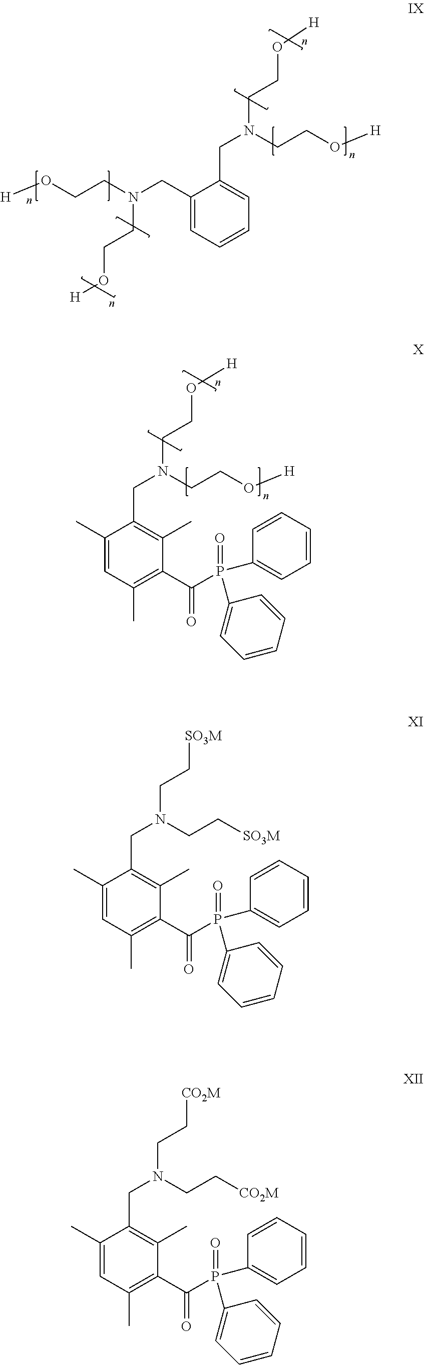

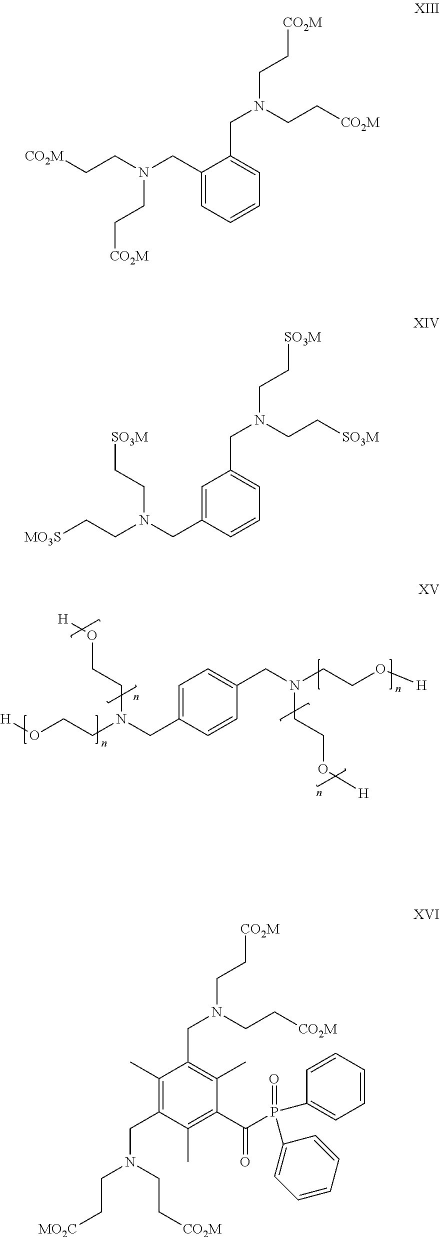

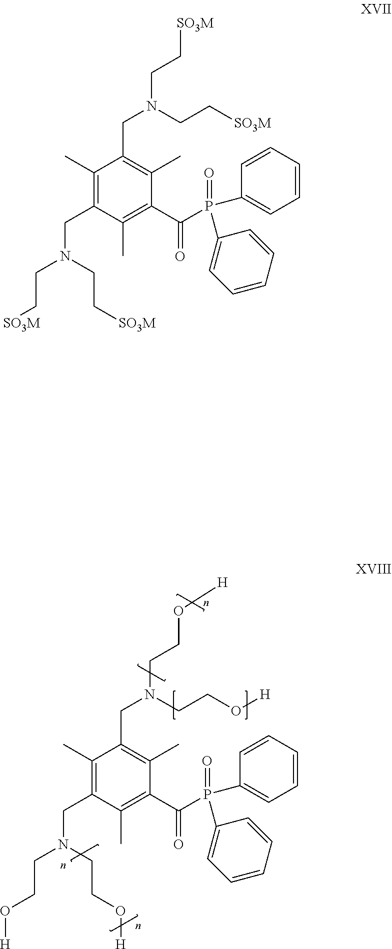

There are various synthetic methods that can be used to generate these water soluble amine synergists. For example, as shown in Scheme 1 below, a reaction can be carried out using commercially available TPO (1) with 3 equiv. of paraformaldehyde in the presence of AlCl₃ in chloroform at 0° C., followed by heating at 60° C., which gives a corresponding TPO chloride (2). Reaction of 2 equiv. of TPO chloride (2) with 1 equiv. of diethanolamine (3) in the presence of K₂CO₃ in acetonitrile under reflux gives a diethanolamine modified water soluble amine synergist of Formula I.

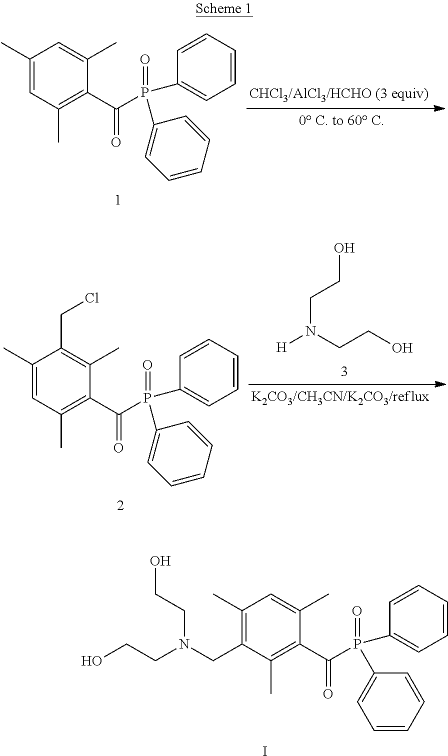

Similarly, as shown in Scheme 2 below, a reaction can be carried out of commercially available TPO (1) with 6 equiv. of paraformaldehyde in the presence of AlCl₃ in chloroform at 0° C., followed by heating at 60° C. This gives a corresponding TPO di-chloride (4). Reaction of TPO di-chloride (4) with 2 equiv of diethanolamine (3) in the presence of K₂CO₃ in acetonitrile under reflux gives the diethanolamine modified water soluble amine synergist of Formula II.

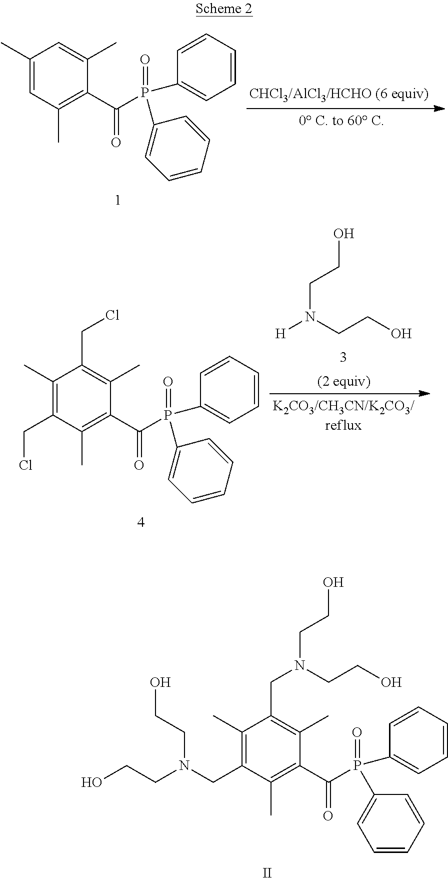

As shown in Scheme 3 below, a reaction can be carried out of commercially available 1,2-bis(chloromethyl)benzene (5) with 2 equiv of diethanolamine (3) in the presence of K₂CO₃ in acetonitrile under reflux to give the diethanolamine modified water soluble amine synergist of Formula III.

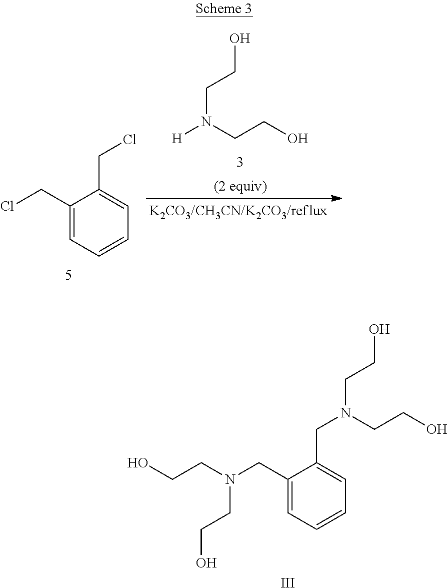 [SMILES: OCCNCCO ; ClCc1ccccc1CCl ; OCCN(CCO)Cc1ccccc1CN(CCO)CCO ; "III"]

As shown in Scheme 4 below, a reaction can be carried out using commercially available 1,3-bis(chloromethyl)benzene (6) with 2 equiv of diethanolamine (3) in the presence of K₂CO₃ in acetonitrile under reflux to give the diethanolamine modified water soluble amine synergist of Formula IV.

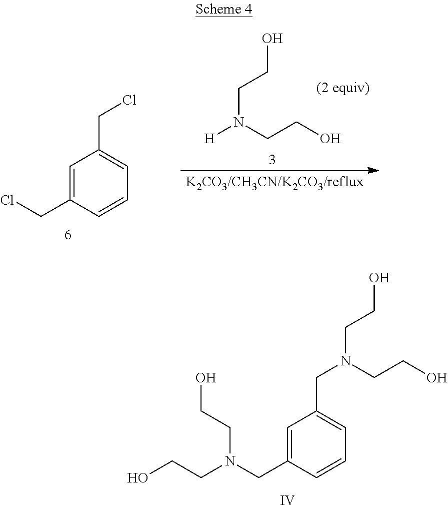

As shown in Scheme 5 below, a reaction of commercially available 1,4-bis(chloromethyl)benzene (7) with 2 equiv of diethanolamine (3) in the presence of K₂CO₃ in acetonitrile under reflux gives the diethanolamine modified water soluble amine synergist of Formula V.

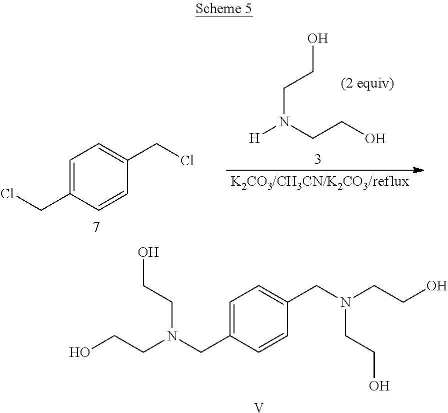

As shown in Scheme 6 below, a reaction of commercially available 1,4-bis(chloromethyl)benzene (7) with 2 equiv of N-(2-carboxyethyl)-β-alanine (8) in the presence of K₂CO₃ in acetonitrile under reflux gives the N-(2-carboxyethyl)-β-alanine amine modified water soluble amine synergist of Formula VI.

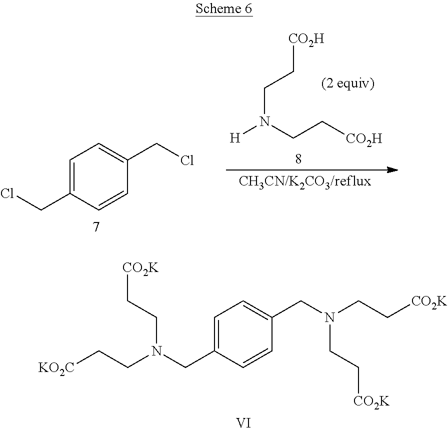

As shown in Scheme 7 below, a reaction of commercially available 1,4-bis(chloromethyl)benzene (7) with 2 equiv of 2,2′-imino-bisethanesulfonic acid (9) in the presence of K₂CO₃ in acetonitrile under reflux gives the 2,2′-imino-bisethanesulfonic acid amine modified water soluble amine synergist of Formula VII.

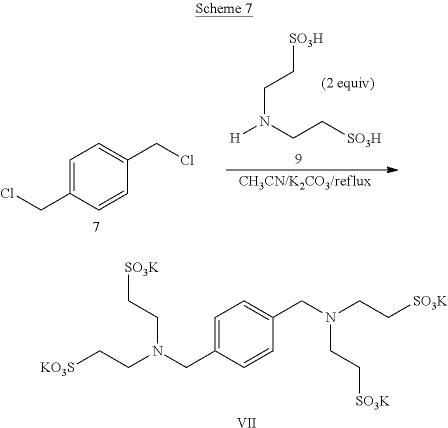

As shown in Scheme 8 below, a reaction of commercially available 1,2-bis(chloromethyl)benzene (5) with 2 equiv of 2,2′-imino-bisethanesulfonic acid (10) in the presence of K₂CO₃ in acetonitrile under reflux gives the 2,2′-imino-bisethanesulfonic acid amine modified water soluble amine synergist of Formula VIII.

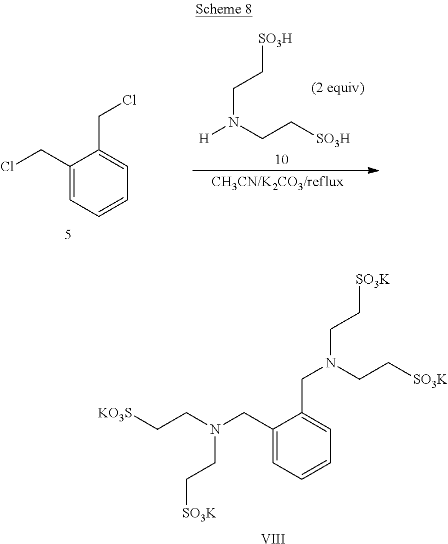

As shown in Scheme 9 below, a reaction of commercially available 1,2-bis(chloromethyl)benzene (5) with 2 equiv of bis(polyethyleneglycol)amine (11) in the presence of K₂CO₃ in acetonitrile under reflux gives the amine modified water soluble amine synergist of Formula IX (where n is from 1 to).

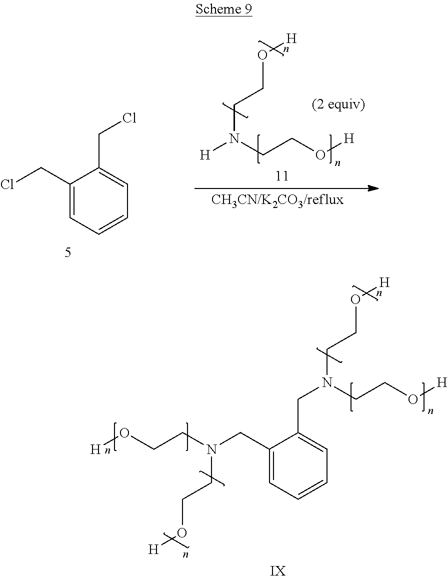

As shown in Scheme 10 below, a Reaction of 1 equiv. of TPO chloride (2) with 1 equiv. of (11) in the presence of K₂CO₃ in acetonitrile under reflux gives a modified water soluble amine synergist of Formula X (where n is from 1 to).

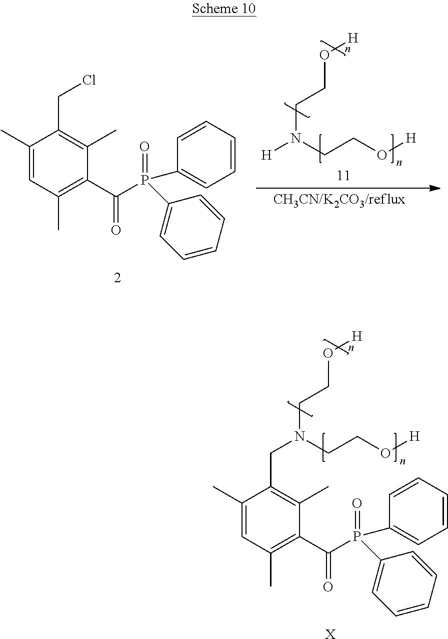

As shown in Scheme 11 below, a reaction of 1 equiv. of TPO chloride (2) with 1 equiv. of (9) in the presence of K₂CO₃ in acetonitrile under reflux gives a modified water soluble amine synergist of Formula XI

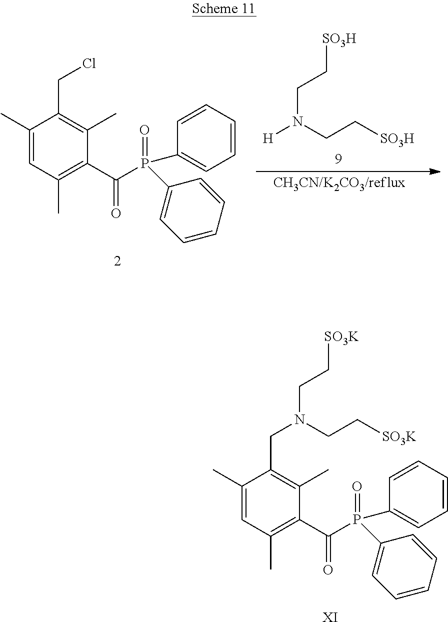

As shown in Scheme 12 below, a reaction of 1 equiv. of TPO chloride (2) with 1 equiv. of c (8) in the presence of K₂CO₃ in acetonitrile under reflux gives a modified water soluble amine synergist of Formula XII.

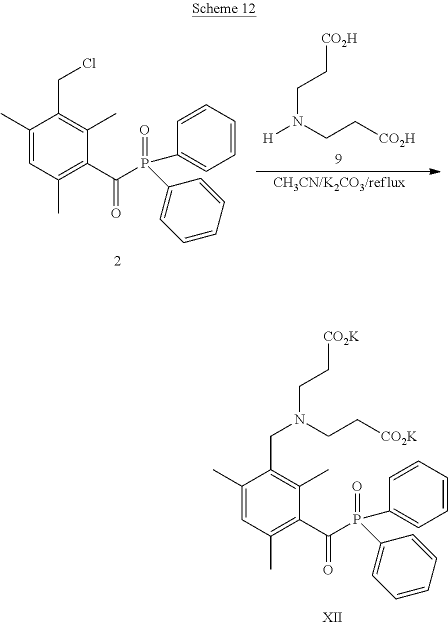

As shown in Scheme 13 below, a reaction can be carried out of commercially available 1,2-bis(chloromethyl)benzene (5) with 2 equiv of c (8) in the presence of K₂CO₃ in acetonitrile under reflux to give the modified water soluble amine synergist of Formula XIII.

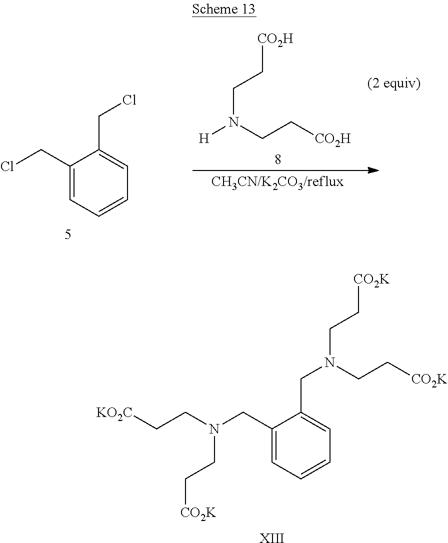

As shown in Scheme 14 below, a reaction can be carried out of commercially available 1,3-bis(chloromethyl)benzene (6) with 2 equiv of (9) in the presence of K₂CO₃ in acetonitrile under reflux to give the modified water soluble amine synergist of Formula XIV.

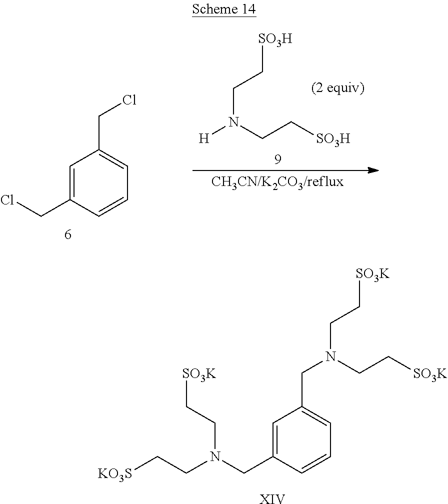

As shown in Scheme 15 below, a reaction of commercially available 1,4-bis(chloromethyl)benzene (7) with 2 equiv of bis(polyethyleneglycol)amine (11) in the presence of K₂CO₃ in acetonitrile under reflux gives the bis(polyethyleneglycol)amine modified water soluble amine synergist of Formula XV (where n is from 1 to 100).

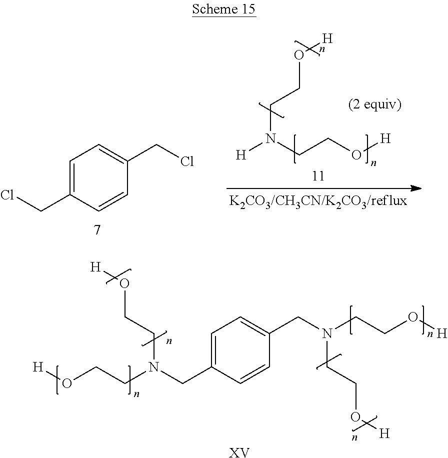

As shown in Scheme 16 below, a reaction of 1 equiv. of di-TPO chloride (4) with 2 equiv. of N-(2-carboxyethyl)-β-alanine (8) in the presence of K₂CO₃ in acetonitrile under reflux gives a modified water soluble amine synergist of Formula XVI.

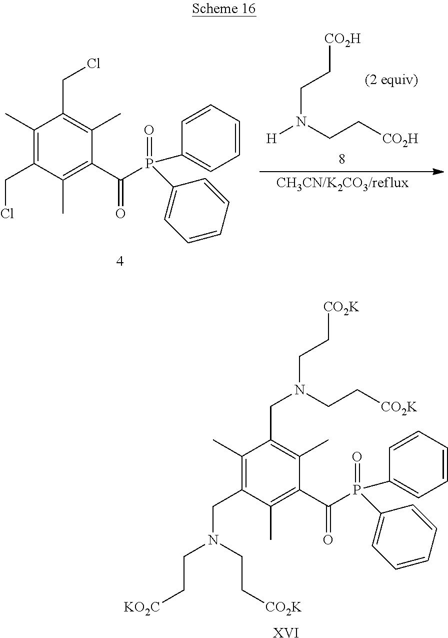

As shown in Scheme 17 below, a reaction of 1 equiv. of di-TPO chloride (4) with 2 equiv. of (9) in the presence of K₂CO₃ in acetonitrile under reflux gives a 2,2′-imino-bisethanesulfonic acid modified water soluble amine synergist of Formula XVII.

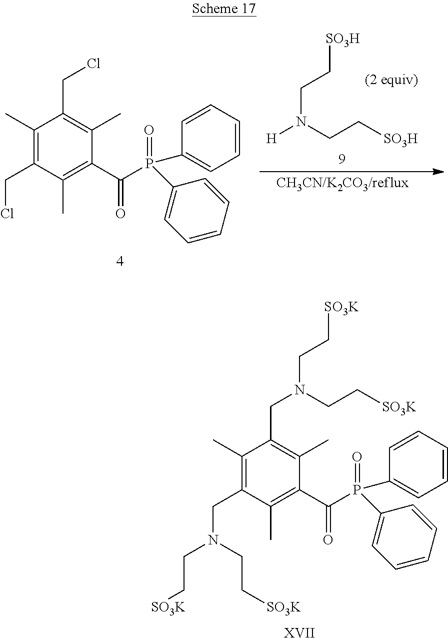

As shown in Scheme 18 below, a reaction of 1 equiv. of di-TPO chloride (4) with 2 equiv. of bis(polyethyleneglycol)amine (11) in the presence of K₂CO₃ in acetonitrile under reflux gives a bis(polyethyleneglycol)amine modified water soluble amine synergist of Formula XVIII (where n is from 1 to 100).

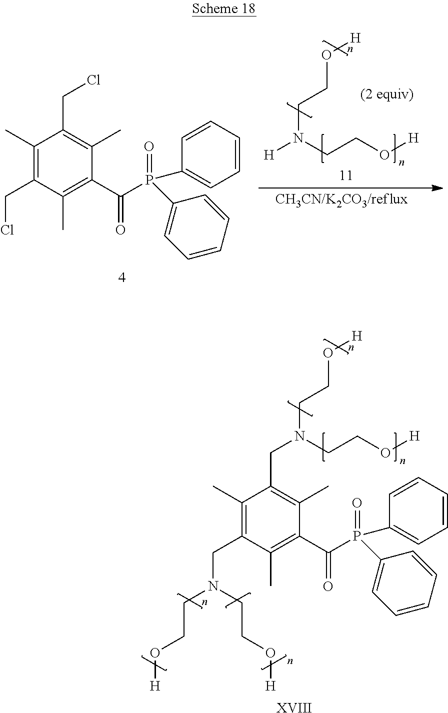

In addition to the water soluble amine synergists described herein, the present disclosure also extends to photo curable inks, such as UV curable inks including UV-LED curable inks. In some examples, a photo curable ink can include a photo reactive binder (such as a UV curable or UV-LED curable binder), a Type II photo initiator, the amine synergist, a colorant, a co-solvent, and water. The amine synergist can be or include one of the water soluble amine synergists shown herein at Formulas I-XVIII, or can include another benzene ring having tertiary amines attached thereto with water soluble end groups.

Typical aqueous ink jet inks can have a pH in the range of 7 to 12. Some commercially available synergists can break down in such basic conditions. Thus, in some examples, the amine synergist described herein can be stable in water and in inks at a pH from 7 to 12, or higher. Thus, the photo curable ink can be formulated to have a pH from 7 to 12, or higher. In some examples, the photo curable ink can have a pH of 8 or higher. In one specific example, the photo curable ink can have a pH of about 8.5. As used herein, “stable” refers to the ability of the amine synergist to have a shelf life of at least 1 year. Typically, aqueous ink jet inks can have a shelf life of greater than 1 year, greater than 2 years, or longer.

In some cases, the photo reactive binder can include a UV or UV-LED curable polyurethane and hydrophobic radiation-curable monomers. In one example, the photo reactive binder can include a waterdispersible (meth)acrylated polyurethane, such as NeoRad® R-441 by NeoResins (Avecia). Other examples of UV reactive binders can include Ucecoat® 7710, Ucecoat® 7655 (available from Cytec), Neorad® R-440, Neorad® R-441, Neorad® R-447, Neorad® R-448 (available from DSM NeoResins), Bayhydrol® UV 2317, Bayhydrol® UV VP LS 2348 (available from Bayer), Lux 430, Lux 399, Lux 484 (available from Alberdingk Boley), Laromer® LR 8949, Laromer® LR 8983, Laromer® PE 22WN, Laromer® PE 55WN, or Laromer® UA 9060 (available from BASF).

The amine synergists of the present disclosure can be used together with a Type II photo initiator, and the combination of Type II photo initiator with the amine synergist can interact by hydrogen abstraction. In this interaction, UV radiation causes a hydrogen radical to be abstracted from the amine synergist onto the Type II photo initiator. This creates two molecules having radicals that can initiate polymerization in the photo reactive binder.

In some cases, the photo curable ink can include two different photo initiators, or a photo initiator and a sensitizer. Some examples of Type II photo initiators can also act as sensitizers. The photo curable ink can also include other polymeric or non-polymeric photo initiators. Examples of radical photo initiators include, by way of illustration and not limitation, 1-hydroxy-cyclohexylphenylketone, benzophenone, 2,4,6-trimethylbenzo-phenone, 4-methylbenzophenone, diphenyl-(2, 4,6-trimethylbenzoyl)phosphine oxide, phenyl bis(2,4,6trimethylbenzoyl) phosphine oxide, 2-hydroxy-2-methyl-1-phenyl-1-propanone, benzyl-dimethyl ketal, 2-methyl-l-[4-(methylthio)phenyl]-2-morpholinopropan-1-one, or combinations thereof. Non-limiting examples of additional photo initiators include alpha amino ketone UV photo initiators such as Ciba® Irgacure® 907, Ciba® Irgacure® 369, and Ciba® Irgacure® 379; bis acylphosphine oxide (BAPO) UV photo initiators such as Irgacure® 819, Darocur® 4265, and Darocur® TPO; alpha hydroxy ketone UV photo initiators such as Irgacure® 184 and Darocur® 1173; including photo initiators with or without sensitizers such as Darocur® ITX (2-isopropyl thioxanthone).

The colorant in the photo curable ink can be a pigment, a dye, or a combination thereof. In some examples, the colorant can be present in an amount from 0.5 wt % to 10 wt % in the photo curable ink. In one example, the colorant can be present in an amount from 1 wt % to 5 wt %. In another example, the colorant can be present in an amount from 5 wt % to 10 wt %.

In some examples, the colorant can be a dye. The dye can be nonionic, cationic, anionic, or a mixture of nonionic, cationic, and/or anionic dyes. Specific examples of dyes that can be used include, but are not limited to, Sulforhodamine B, Acid Blue 113, Acid Blue 29, Acid Red 4, Rose Bengal, Acid Yellow 17, Acid Yellow 29, Acid Yellow 42, Acridine Yellow G, Acid Yellow 23, Acid Blue 9, Nitro Blue Tetrazolium Chloride Monohydrate or Nitro BT, Rhodamine 6G, Rhodamine 123, Rhodamine B, Rhodamine B Isocyanate, Safranine O, Azure B, and Azure B Eosinate, which are available from Sigma-Aldrich Chemical Company (St. Louis, Mo.). Examples of anionic, water-soluble dyes include, but are not limited to, Direct Yellow 132, Direct Blue 199, Magenta 377 (available from Ilford AG, Switzerland), alone or together with Acid Red 52. Examples of water-insoluble dyes include azo, xanthene, methine, polymethine, and anthraquinone dyes. Specific examples of water-insoluble dyes include Orasol® Blue GN, Orasol® Pink, and Orasol® Yellow dyes available from Ciba-Geigy Corp. Black dyes may include, but are not limited to, Direct Black 154, Direct Black 168, Fast Black 2, Direct Black 171, Direct Black 19, Acid Black 1, Acid Black 191, Mobay Black SP, and Acid Black 2.

In other examples, the colorant can be a pigment. The pigment can be self-dispersed with a polymer, oligomer, or small molecule; or can be dispersed with a separate dispersant. Suitable pigments include, but are not limited to, the following pigments available from BASF: Paliogen® Orange, Heliogen® Blue L 6901F, Heliogen® Blue NBD 7010, Heliogen® Blue K 7090, Heliogen® Blue L 7101F, Paliogen® Blue L 6470, Heliogen® Green K 8683, and Heliogen® Green L 9140. The following black pigments are available from Cabot: Monarch® 1400, Monarch® 1300, Monarch® 1100, Monarch® 1000, Monarch® 900, Monarch® 880, Monarch® 800, and Monarch® 700. The following pigments are available from CIBA: Chromophtal® Yellow 3G, Chromophtal® Yellow GR, Chromophtal® Yellow 8G, Igrazin® Yellow 5GT, Igralite® Rubine 4BL, Monastral® Magenta, Monastral® Scarlet, Monastral® Violet R, Monastral® Red B, and Monastral® Violet Maroon B. The following pigments are available from Degussa: Printex® U, Printex® V, Printex® 140U, Printex® 140V, Color Black FW 200, Color Black FW 2, Color Black FW 2V, Color Black FW 1, Color Black FW 18, Color Black S 160, Color Black S 170, Special Black 6, Special Black 5, Special Black 4A, and Special Black 4. The following pigment is available from DuPont: Tipure® R-101. The following pigments are available from Heubach: Dalamar® Yellow YT-858-D and Heucophthal Blue G XBT-583D. The following pigments are available from Clariant: Permanent Yellow GR, Permanent Yellow G, Permanent Yellow DHG, Permanent Yellow NCG-71, Permanent Yellow GG, Hansa Yellow RA, Hansa Brilliant Yellow 5GX-02, Hansa Yellow-X, Novoperm® Yellow HR, Novoperm® Yellow FGL, Hansa Brilliant Yellow 10GX, Permanent Yellow G3R-01, Hostaperm® Yellow H4G, Hostaperm® Yellow H3G, Hostaperm® Orange GR, Hostaperm® Scarlet GO, and Permanent Rubine F6B. The following pigments are available from Mobay: Quindo® Magenta, Indofast® Brilliant Scarlet, Quindo® Red R6700, Quindo® Red R6713, and Indofast® Violet. The following pigments are available from Sun Chemical: L74-1357 Yellow, L75-1331 Yellow, and L75-2577 Yellow. The following pigments are available from Columbian: Raven® 7000, Raven® 5750, Raven® 5250, Raven® 5000, and Raven® 3500. The following pigment is available from Sun Chemical: LHD9303 Black. Any other pigment and/or dye can be used that is useful in modifying the color of the UV curable ink. Additionally, the colorant can include a white pigment such as titanium dioxide, or other inorganic pigments such as zinc oxide and iron oxide.

The components of the photo curable ink can be selected to give the ink good ink jetting performance. Besides the photo curable binder, amine synergist, photo initiator, and the colorant, the photo curable ink can also include a liquid vehicle. Liquid vehicle formulations that can be used in the photo curable ink can include water and one or more co-solvents present in total at from 1 wt % to 50 wt %, depending on the jetting architecture. Further, one or more non-ionic, cationic, and/or anionic surfactant can be present, ranging from 0.01 wt % to 20 wt %. In one example, the surfactant can be present in an amount from 5 wt % to 20 wt %. The liquid vehicle can also include dispersants in an amount from 5 wt % to 20 wt %. The balance of the formulation can be purified water, or other vehicle components such as biocides, viscosity modifiers, materials for pH adjustment, sequestering agents, preservatives, and the like. In one example, the liquid vehicle can be predominantly water, e.g., greater than 50 wt % water.

With respect to the classes of co-solvents that can be used, these compounds can include organic co-solvents including aliphatic alcohols, aromatic alcohols, diols, glycol ethers, polyglycol ethers, caprolactams, formamides, acetamides, and long chain alcohols. Examples of such compounds include primary aliphatic alcohols, secondary aliphatic alcohols, 1,2-alcohols, 1,3-alcohols, 1,5-alcohols, ethylene glycol alkyl ethers, propylene glycol alkyl ethers, higher homologs (C₆-C₁₂) of polyethylene glycol alkyl ethers, N-alkyl caprolactams, unsubstituted caprolactams, both substituted and unsubstituted formamides, both substituted and unsubstituted acetamides, and the like. Specific examples of solvents that can be used include, but are not limited to, 2-pyrrolidinone, N-methylpyrrolidone, 2-hydroxyethyl-2-pyrrolidone, 2-methyl-1,3-propanediol, tetraethylene glycol, 1,6-hexanediol, 1,5-hexanediol and 1,5-pentanediol.

With respect to the surfactants that can be used, one or more example surfactant that can be used includes, alkyl polyethylene oxides, alkyl phenyl polyethylene oxides, polyethylene oxide block copolymers, acetylenic polyethylene oxides, polyethylene oxide (di)esters, polyethylene oxide amines, protonated polyethylene oxide amines, protonated polyethylene oxide amides, dimethicone copolyols, substituted amine oxides, and the like. As mentioned, the amount of surfactant added to the formulation of this disclosure may range from 0.01 wt % to 20 wt %, if present. Suitable surfactants can include, but are not limited to, liponic esters such as Tergitol™ 15-S-12, Tergitol™ 15-S-7 available from Dow Chemical Company, LEG-1 and LEG-7; Triton™ X-100; Triton™ X-405 available from Dow Chemical Company; LEG-1, and sodium dodecylsulfate.

Consistent with the formulation of this disclosure, various other additives can be employed to optimize the properties of the ink composition for specific applications. Examples of these additives are those added to inhibit the growth of harmful microorganisms. These additives may be biocides, fungicides, and other microbial agents, which are routinely used in ink formulations. Examples of suitable microbial agents include, but are not limited to, NUOSEPT® (Nudex, Inc.), UCARCIDE™ (Union carbide Corp.), VANCIDE® (R.T. Vanderbilt Co.), PROXEL® (ICI America), and combinations thereof.

Sequestering agents, such as EDTA (ethylene diamine tetra acetic acid), may be included to eliminate the deleterious effects of heavy metal impurities, and buffer solutions may be used to control the pH of the ink. From 0.01 wt % to 2 wt %, for example, can be used. Viscosity modifiers and buffers may also be present, as well as other additives to modify properties of the ink as desired. Such additives can be present at from 0.01 wt % to 20 wt %.

Table A shows the composition of an example of a photo curable ink formulation (e.g. UV LED curable) in accordance with the present disclosure. The ink can be formulated by mixing these ingredients or by other formulations. The pH of the ink can then be adjusted. In one example, the ingredients can be stirred for 30 minutes, and then aqueous potassium hydroxide can be added to adjust the pH to 8.5. It is noted that though water concentrations are listed as “balance,” it is understood that the balance of components could include other liquid vehicle components or minor amounts of solids often present in inkjet ink compositions.

TABLE A

| Component | Weight Percent |
|---|---|
| Photo reactive binder | 1-20% |
| Type II Photo initiator | 0.15-5% |
| Co-photo initiator | 0-10% |
| Amine synergist | 0.1-5% |
| Surfactant | 0-10% |
| Anti-kogation agent | 0-5% |
| Colorant | 0.5-10% |
| Organic Co-solvent | 0.1-50% |
| Water | Balance |

The photo curable ink can be used to print on a broad selection of substrates including untreated plastics, flexible as well as rigid, porous substrates such as paper, cardboard, foam board, textile, and others. The ink has a good adhesion on a variety of substrates. The photo curable ink also has a good viscosity, enabling good printing performances and enables the ability to formulate inks suitable for inkjet application. In some examples, the ink can be formulated for thermal inkjet printing. The photo-curable ink composition of the present disclosure enables high printing speed and is very well suited for use in digital inkjet printing.

The present disclosure also extends to a method of making a photo curable ink, as shown in FIG. 2. In an example, a method can include mixing 210 a photo reactive binder, a Type II photo initiator, a colorant, and a liquid vehicle including co-solvent and water with a tertiary benzylamine including a substituted benzene ring attached to a tertiary amine. In this example, the tertiary amine can also be modified with multiple water soluble groups. The water soluble groups can be one or more of C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups. In one example, the substituted benzene ring is provided by a diphenyl(2,4,6-trimethyl benzoyl)phosphine oxide.

It is to be understood that this disclosure is not limited to the particular process steps and materials disclosed herein because such process steps and materials may vary somewhat. It is also to be understood that the terminology used herein is used for the purpose of describing particular examples only. The terms are not intended to be limiting because the scope of the present disclosure is intended to be limited only by the appended claims and equivalents thereof.

It is noted that, as used in this specification and the appended claims, the singular forms “a,” “an,” and “the” include plural referents unless the context clearly dictates otherwise.

As used herein, “UV curable” refers to compositions that can be cured by exposure to ultraviolet light from any UV source such as a mercury vapor lamp, UV LED source, or the like. Mercury vapor lamps emit high intensity light at wavelengths from 240 nm to 270 nm and 350 nm to 380 nm. “LED curable” refers to compositions that can be cured by ultraviolet light from an ultraviolet LED. Ultraviolet LEDs typically emit light at specific wavelengths. For example, ultraviolet LEDs are available at 365 nm and 395 nm wavelengths, among others. The term “photo curable” refers generally to compositions that can be cured by exposure to light from any wavelength suitable for the composition being cured. Typically, the photo curable composition will be UV curable, and in some cases UV LED curable.

As used herein, “liquid vehicle” or “ink vehicle” refers to a liquid fluid in which colorant is placed to form an ink. A wide variety of ink vehicles may be used with the systems and methods of the present disclosure. Such ink vehicles may include a mixture of a variety of different agents, including surfactants, solvents, co-solvents, anti-kogation agents, buffers, biocides, sequestering agents, viscosity modifiers, surface-active agents, water, etc.

As used herein, “colorant” can include dyes and/or pigments.

As used herein, “dye” refers to compounds or molecules that absorb electromagnetic radiation or certain wavelengths thereof. Dyes can impart a visible color to an ink if the dyes absorb wavelengths in the visible spectrum.

As used herein, “pigment” generally includes pigment colorants, magnetic particles, aluminas, silicas, and/or other ceramics, organo-metallics or other opaque particles, whether or not such particulates impart color. Thus, though the present description primarily exemplifies the use of pigment colorants, the term “pigment” can be used more generally to describe not only pigment colorants, but other pigments such as organometallics, ferrites, ceramics, etc. In one specific example, however, the pigment is a pigment colorant.

As used herein, “ink-jetting” or “jetting” refers to compositions that are ejected from jetting architecture, such as ink-jet architecture. Ink-jet architecture can include thermal or piezo architecture. Additionally, such architecture can be configured to print varying drop sizes such as less than 10 picoliters, less than 20 picoliters, less than 30 picoliters, less than 40 picoliters, less than 50 picoliters, etc.

As used herein, the term “substantial” or “substantially” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, the term “about” is used to provide flexibility to a numerical range endpoint by providing that a given value may be “a little above” or “a little below” the endpoint. The degree of flexibility of this term can be dictated by the particular variable and determined based on the associated description herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include individual numerical values or sub-ranges encompassed within that range as if each numerical value and sub-range is explicitly recited. As an illustration, a numerical range of “about 1 wt % to about 5 wt %” should be interpreted to include not only the explicitly recited values of about 1 wt % to about 5 wt %, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

### EXAMPLES

The following illustrates several examples of the present disclosure. However, it is to be understood that the following are only illustrative of the application of the principles of the present disclosure. Numerous modifications and alternative compositions, methods, and systems may be devised without departing from the spirit and scope of the present disclosure. The appended claims are intended to cover such modifications and arrangements.

### Example 1—Synthesis of TPO Chloride (2)

A mixture of 5.0 grams (14.3 mmol) of commercially available TPO (1) in 100 mL of chloroform was cooled to 0° C. under N₂ and, under mechanical stirring, AlCl₃ (12.0 grams, 85.8 mmol) and paraformaldehyde (1.3 grams, 43 mmol) was added portion-wise. After the addition, the mixture was heated to 60° C. for 6 hours. Then, the reaction mixture was cooled to room temperature and poured into ice-water. The mixture was extracted with dichloromethane (3×100 mL). The combined organic layers were washed by water, brine, and then dried over sodium sulfate. Evaporation of solvent gave a residue, which was further purified by flash chromatography using hexanes/ethyl acetate (100% hexanes to 50% hexanes in ethyl acetate) as eluents, giving rise to 3.7 grams (65 wt %) of the desired TPO chloride (2).

### Example 2—Synthesis of TPO Di-Chloride (4)

A mixture of 5.0 grams (14.3 mmol) of commercially available TPO (1) in 100 mL of chloroform was cooled to 0° C. under N₂ and, under mechanical stirring, AlCl₃ (12.0 grams, 85.8 mmol) and paraformaldehyde (2.6 grams, 86 mmol) were added portion-wise. After the completion of the addition, the mixture was heated to 60° C. for 12 hours. Then, the reaction mixture was cooled to room temperature and poured into ice-water. The mixture was extracted with dichloromethane (3×100 mL). The combined organic layers were washed by water, brine, and then dried over sodium sulfate. Evaporation of solvent gave a residue, which was further purified by flash chromatography using hexanes/ethyl acetate (100% hexanes to 50% hexanes in ethyl acetate) as eluents, giving rise to 3.8 grams (60 wt %) of the desired TPO-dichloride (4).

### Example 3—Synthesis of Water Soluble Amine Synergist (Formula I)

To a solution of TPO chloride (2) (7.94 grams, 20 mmol) in 150 mL of acetonitrile was added potassium carbonate (3.03 grams, 22 mmol). To this mixture was added a solution of diethanolamine (3) (2.31 grams, 22 mmol) in 50 mL of acetonitrile. After the completion of the addition, the reaction mixture was stirred under reflux for 24 hours. Then, the reaction mixture was cooled to room temperature. The solid was filtered off by filtration and washed with ethyl acetate. The combined organic layers were washed by water, brine and then dried over sodium sulfate. Evaporation of solvent gave a residue, which was further purified by flash chromatography using hexanes/ethyl acetate (100% hexanes to 50% hexanes in ethyl acetate) as eluents, giving rise to 8.0 grams (86 wt %) of the water soluble amine synergist of Formula I.

### Example 4—Synthesis of Water Soluble Amine Synergist (Formula II)

To a solution of TPO di-chloride (4) (8.90 grams, 20 mmol) in 150 mL of acetonitrile was added potassium carbonate (6.07 grams, 44 mmol). To the above mixture was added a solution of diethanolamine (3) (4.62 grams, 44 mmol) in 50 mL of acetonitrile. After the completion of the addition, the reaction mixture was stirred under reflux for 24 hours. Then, the reaction mixture was cooled to room temperature. The solid was filtered off by filtration and washed with ethyl acetate. The combined organic layers were washed by water, brine and then dried over sodium sulfate. Evaporation of solvent gave a residue, which was further purified by flash chromatography using hexanes/ethyl acetate (100% hexanes to 50% hexanes in ethyl acetate) as eluents, giving rise to 3.78 grams (65 wt %) of the water soluble amine synergist of Formula II.

### Example 5—Synthesis of Water Soluble Amine Synergist (Formula III)

To a solution of 1,2-bis(chloromethyl)benzene (5) (17.5 grams, 100 mmol) in 300 mL of acetonitrile was added potassium carbonate (30.36 grams, 220 mmol). To the above mixture was added a solution of diethanolamine (3) (23.1 grams, 220 mmol) in 50 mL of acetonitrile. After the completion of the addition, the reaction mixture was stirred under reflux for 24 hours. Then, the reaction mixture was cooled to room temperature. The solid was filtered off by filtration and washed with ethyl acetate. The combined organic layers were washed by water, brine and then dried over sodium sulfate. Evaporation of solvent gave a residue, which was further purified by flash chromatography using hexanes/ethyl acetate (100% hexanes to 50% hexanes in ethyl acetate) as eluents, giving rise to 26.83 grams (86 wt %) of the water soluble amine synergist of Formula III.

### Example 6—Synthesis of Water Soluble Amine Synergist (Formula IV)

To a solution of 1,3-bis(chloromethyl)benzene (6) (17.5 grams, 100 mmol) in 300 mL of acetonitrile was added potassium carbonate (30.36 grams, 220 mmol). To the above mixture was added a solution of diethanolamine (3) (23.1 grams, 220 mmol) in 50 mL of acetonitrile. After the completion of the addition, the reaction mixture was stirred under reflux for 24 hours. Then, the reaction mixture was cooled to room temperature. The solid was filtered off by filtration and washed with ethyl acetate. The combined organic layers were washed by water, brine and then dried over sodium sulfate. Evaporation of solvent gave a residue, which was further purified by flash chromatography using hexanes/ethyl acetate (100% hexanes to 50% hexanes in ethyl acetate) as eluents, giving rise to 28.08 grams (90 wt %) of the water soluble amine synergist of Formula IV.

### Example 7—Synthesis of Water Soluble Amine Synergist (Formula V)

To a solution of 1,4-bis(chloromethyl)benzene (7) (17.5 grams, 100 mmol) in 300 mL of acetonitrile was added potassium carbonate (30.36 grams, 220 mmol). To the above mixture was added a solution of diethanolamine (3) (23.1 grams, 220 mmol) in 50 mL of acetonitrile. After the completion of the addition, the reaction mixture was stirred under reflux for 24 hours. Then, the reaction mixture was cooled to room temperature. The solid was filtered off by filtration and washed with ethyl acetate. The combined organic layers were washed by water, brine and then dried over sodium sulfate. Evaporation of solvent gave a residue, which was further purified by flash chromatography using hexanes/ethyl acetate (100% hexanes to 50% hexanes in ethyl acetate) as eluents, giving rise to 27.77 grams (89%) of the desired water soluble amine synergist V.

### Example 8—Photo Curable Inkjet Inks 1-5

Several photo (UV-LED) curable inkjet ink were prepared by mixing the following components as shown in Table 1.

TABLE 1

| | Ink 1 | Ink 2 | Ink 3 | Ink 4 | Ink 5 |
|---|---|---|---|---|---|
| Component | (wt %) | (wt %) | (wt %) | (wt %) | (wt %) |
| UV reactive binder | 15 | 5 | 10 | 15 | 10 |
| Irgacur ® 819 | 0.3 | — | — | 0.3 | — |
| (co-photo initiator | | | | | |
| from BASF) | | | | | |
| Thioxanthone derivative | 0.5 | 0.25 | 0.25 | 0.5 | 0.25 |
| PEG-600 (Type II | | | | | |
| photo initiator) | | | | | |
| Water soluble amine | 0.5 | 0.5 | 0.5 | 1 | 0.5 |
| synergist (I, II, III, | (I) | (II) | (III) | (IV) | (V) |
| IV, or V) | | | | | |
| LEG-1 ® (surfactant) | 1 | 1 | 1 | 1 | 1 |
| Surfynol ® CT-211 | 1 | 0.5 | 0.5 | 1 | 0.5 |
| (surfactant from | | | | | |
| Air Products) | | | | | |
| Crodafos ® N3 | 0.5 | 0.5 | 0.5 | 0.5 | 0.5 |
| (anti-kogation agent | | | | | |
| from Croda) | | | | | |
| Pigments | 2.5 | 3 | 3 | 2.5 | 3 |
| 2-hydroxyethyl-2- | 10 | 10 | 10 | 10 | 10 |
| pyrrolidone (co-solvent) | | | | | |
| Water | 69.2 | 79.15 | 74.15 | 68.7 | 74.15 |

### Example 17—Preparation of Photo Curable Ink 1

Photo Curable Ink 1 was prepared in accordance with the following steps. Notably, Photo Curable Inks 2-5 can be prepared using similar steps. As a first batch, a UV reactive binder was mixed with a minor portion of the total water (less than about 30 wt %) and the Irgacure 819 (co-photo initiator) at 60° C. for 5 minutes. As a second batch, 2-hydroxyethyl-2-pyrrolidone (co-solvent) was mixed with a larger portion of the total water content (less than about 70 wt %) and Crodafos® N3A, CT211, and LEG-1. The second batch was neutralized to a pH of 7.5 with KOH solution. The first batch and the second batch were then combined. Next, a thioxanthone derivative of PEG-600 (sensitizer) and the water soluble TPO (photo initiator I) were added and mixed well until they dissolved into the mixture. The black pigment dispersion was then added to the admixture and the pH was adjusted to 8.5 using KOH solution. Notably, some additional water content is added during subsequent steps after combining the first and second batch, resulting in the water content listed in Table 1.

A print durability test was then conducted using Photo Curable Ink 1, as follows:

Sample Preparation
1. The Photo Curable Ink was filled into a thermal inkjet pen.
2. Fixer was printed from a different pen right before the ink was printed. The fixer formulation was a solution of calcium propionate, calcium nitrate tetrahydrate, Surfynol® SEF, tetraethylene glycol, and water.
3. Two paper substrates were evaluated:
   a. Offset coated paper—Sterling® Ultra Gloss (SUG), and
   b. White Top coated Kraft Liner—RockTenn® 1 (RT1).
4. The ink was immediately dried using a hot air blower for 5 seconds at 375° F.
5. The dried ink was then immediately cured at a speed of 100 feet per minute using a 16 W/cm² LED 395 nm wavelength (from Phoseon).

Durability Measurements
1. Wet Rub Test
   1. This test was performed after a pre-defined time period after printing and curing. For SUG it was performed 24 hours after printing, and for RT1 it was performed 72 hours after printing.
   2. A taber test tool was used with Crockmeier cloth attached to the tip.
   3. The weight load was 350 gram.
   4. One (1) cycle was used for SUG, and two (2) cycles was used for RT1.
   5. The solution was Windex®;
   6. For the Wet Rub Test, the delta optical density (ΔOD) was measured before and after the rub; and
   7. In this test, the lower the ΔOD, the better the durability.
   8. A ΔOD<0.15 is considered to be a good score.
2. Immediate Rub Test
   1. Hand held rubbing tool was used to assess the smearing of dried and cured ink immediately after printing;
   2. The tool used was fit with a rubber tip that when pushed down to apply a constant pressure of 6 to 7 lbs;
   3. For the Immediate Rub Test, the delta optical density (ΔOD) was measured before and after the rub.
   4. In this test, the lower the ΔOD, the better the durability.
   5. A ΔOD<0.15 is considered a good score.

The Results for the Wet Rub Test and the Immediate Rub Test are provided in Table 2. Notably, the black Photo Curable Ink was printed as described and tested both with and without curing on both papers (SUG and RT1).

TABLE 2
Results for Photo Curable Black Ink 1

| | With Curing | | Without Curing | |
|---|---|---|---|---|
| | Wet | Immediate | Wet | Immediate |
| | Rub | Rub | Rub | Rub |
| Paper | (ΔOD) | (ΔOD) | (ΔOD) | (ΔOD) |
| SUG | 0.12 | 0.18 | 1.89 | 0.95 |
| RTI | 0.05 | 0.07 | 1.45 | 0.75 |

As can be seen in Table 1 above, the black Photo Curable Ink exhibited significantly better wet rub and immediate rub resistance after curing. The initial OD was 2.08 and 1.76 on SUG and RT1, respectively, and therefore a ΔOD of 0.12, for example, indicates that after rubbing the print, only 0.12 OD units were lost from the initial 2.08 OD measurement. Conversely, without curing, a ΔOD of 1.89 means that that the ink lost significant OD units from the initial 2.08 OD measurement. The durability improvement by curing is evident in both Wet Rub and Immediate Rub measurements, suggesting that the polymeric TPO based photo initiator package efficiently participated in the curing and crosslinking of the Photo Curable Ink.

While the present technology has been described with reference to certain examples, those skilled in the art will appreciate that various modifications, changes, omissions, and substitutions can be made without departing from the spirit of the disclosure. It is intended, therefore, that the disclosure be limited only by the scope of the following claims.

What is claimed is:

1. An amine synergist, comprising a tertiary benzylamine including a substituted benzene ring attached to a tertiary amine, wherein the tertiary amine is modified with multiple water soluble groups.

2. The amine synergist of claim 1, wherein one or more of the water soluble groups is C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups.

3. The amine synergist of claim 1, wherein the substituted benzene ring is provided by a diphenyl(2,4,6-trimethylbenzoyl)phosphine oxide.

4. The amine synergist of claim 1, having a general formula: 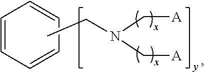
wherein A is independently a water solubilizing group, x is independently from 2 to 5, and y is from 1 to 4.

5. The amine synergist of claim 4, wherein A is hydroxyl, sulfonate, sulfonic acid, carboxylate, carboxylic acid, or polyethylene glycol with from 2 to 20 ether groups.

6. The amine synergist of claim 1, having the general formula: 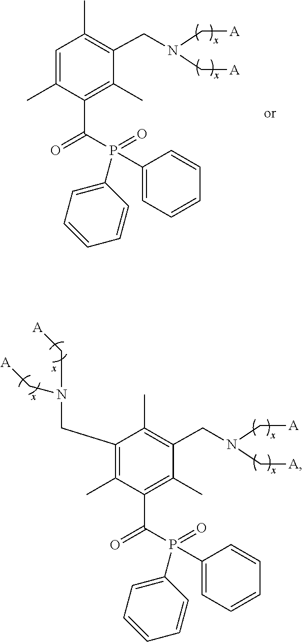
wherein A is independently a water solubilizing group, and x is independently from 2 to 5.

7. The amine synergist of claim 6, wherein A is hydroxyl, sulfonate, sulfonic acid, carboxylate, carboxylic acid, or polyethylene glycol with from 2 to 20 ether groups.

8. The amine synergist of claim 1, having one of the following structures: 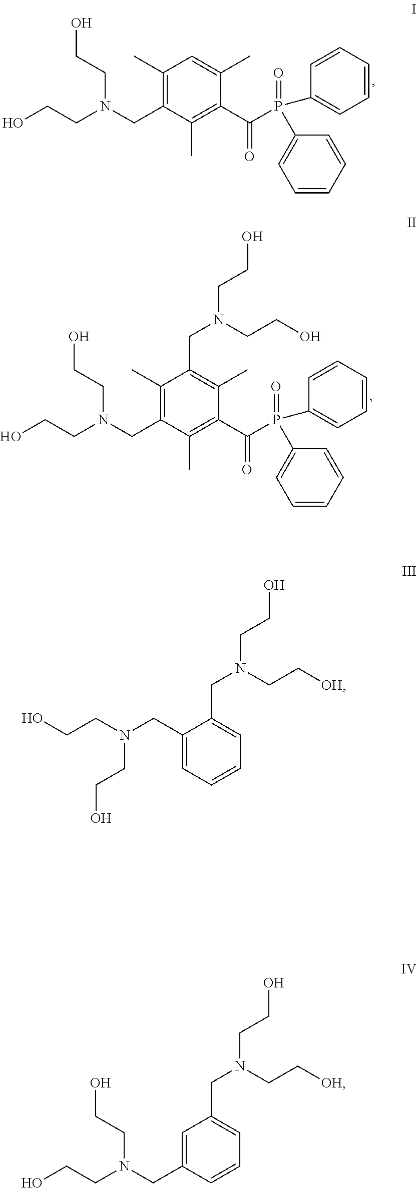 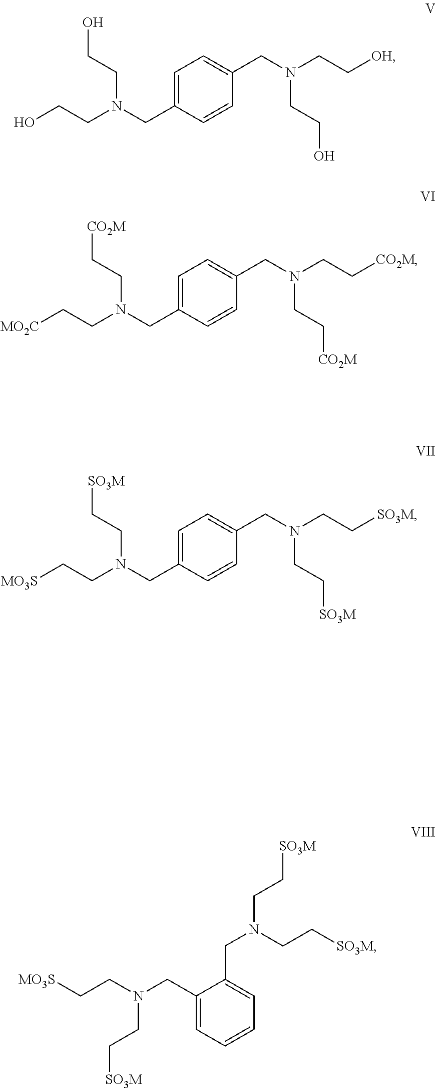 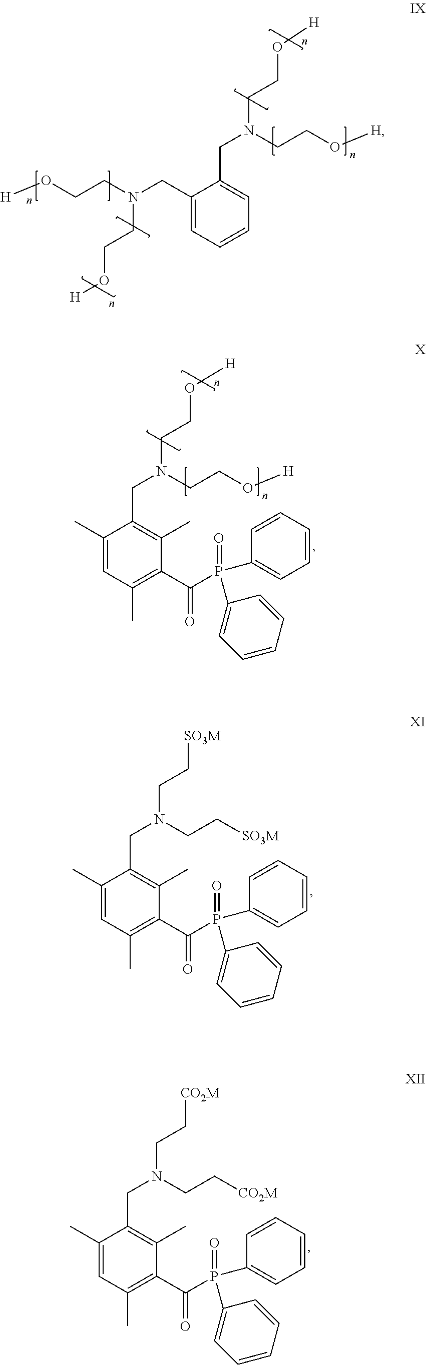 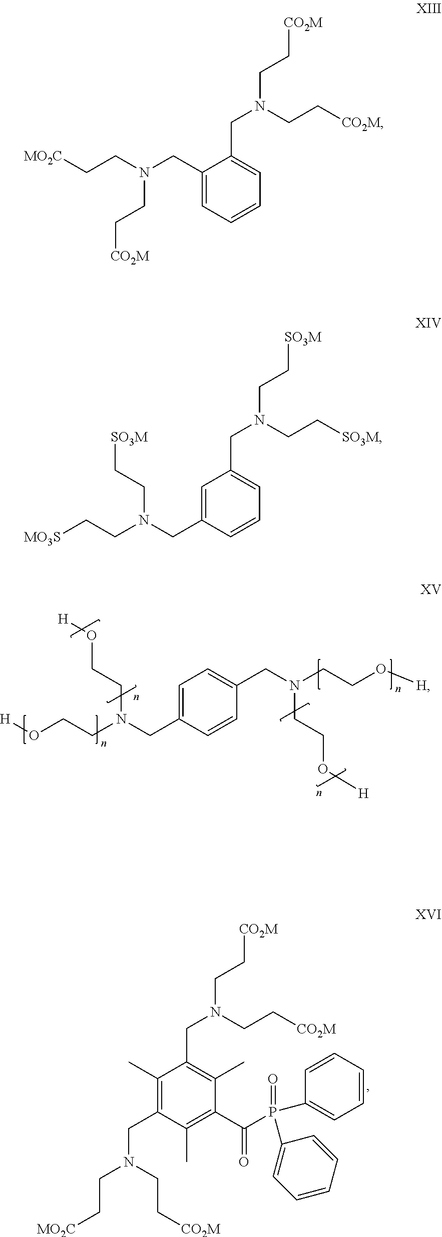 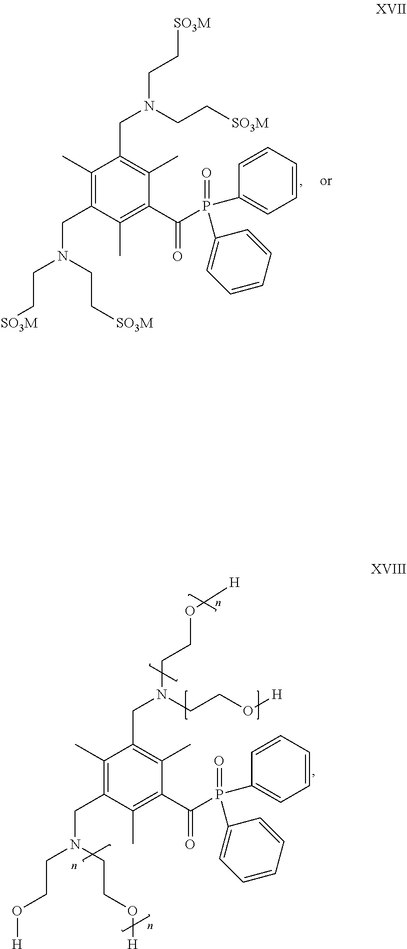
wherein n is from 1 to 100; and M is hydrogen or a monovalent cation.

9. A photo curable ink, comprising:
a photo reactive binder;
an amine synergist comprising a tertiary benzylamine including a substituted benzene ring attached to a tertiary amine, wherein the tertiary amine is modified with multiple water soluble groups;
a Type II photo initiator;
a colorant; and
a liquid vehicle including co-solvent and water.

10. The photo curable ink of claim 9, wherein the photo curable ink has a pH from 7 to 12 and the amine synergist is stable in the photo curable ink.

11. The photo curable ink of claim 9, wherein the Type II photo initiator is a polymeric photo initiator and wherein the photo curable ink further comprises a co-photo initiator.

12. The photo curable ink of claim 9, wherein the wherein one or more of the water soluble groups of the amine synergist is C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups.

13. The photo curable ink of claim 9, wherein the substituted benzene ring is provided by a diphenyl(2,4,6-trimethylbenzoyl)phosphine oxide.

14. A method of making a photo curable ink, comprising mixing a photo reactive binder, a Type II photo initiator, a colorant, and a liquid vehicle including co-solvent and water with a tertiary benzylamine including a substituted benzene ring attached to a tertiary amine, wherein the tertiary amine is also modified with multiple water soluble groups.

15. The method of claim 14, wherein the wherein one or more of the water soluble groups of the amine synergist is C2 to C5 alkyl hydroxyl, C2 to C5 alkyl sulfonate, C2 to C5 alkyl sulfonic acid, C2 to C5 alkyl carboxylate, C2 to C5 alkyl carboxylic acid, or polyethylene glycol with from 2 to 20 ether groups.
